(19) United States
(12) Patent Application Publication
(10) Pub. No.: US 2007/0259920 A1
(43) Pub. Date: Nov. 8, 2007

(54) P2X7, receptor antagonists and uses thereof

(76) Inventors: William A. Carroll, Evanston, IL (US); Arturo Perez-Medrano, Grayslake, IL (US); Tongmei Li, Lake Bluff, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/593,773

(22) Filed: Nov. 7, 2006

Related U.S. Application Data
(60) Provisional application No. 60/734,938, filed on Nov. 9, 2005.

(51) Int. Cl. A61K 31/415; A61K 31/4427; A61P 25/28; A61P 29/00; C07D 231/10

(52) U.S. Cl. 514/336; 514/406; 546/275.7; 548/358.1; 548/373.1; 548/377.1

(57) ABSTRACT

A compound having formula (I) 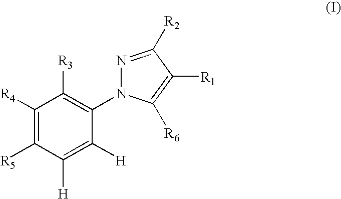
wherein R₁, R₂, R₃, R₄, R₅ and R₆ are defined in the description, is disclosed as an P2X₇ antagonist. Methods and compositions for treating disease or condition modulated by P2X₇ are also disclosed.

This application claims priority to the provisional application Ser. No. 60/734,938 filed on Nov. 9, 2005.

### TECHNICAL FIELD

The present invention relates to compounds of formula (I) that are P2X₇ receptor antagonists and their use for treating pain, neuropathic pain, inflammation, rheumatoid arthritis, neurodegeneration, depression and for promoting neuroregeneration.

### BACKGROUND OF THE INVENTION

P2X receptors are ionotropic receptors activated by ATP. The importance of P2X receptors in nociception is underscored by the variety of pain states in which this endogenous ligand can be released. Of the seven P2X receptors, the P2X₇ is distinguished by its ability to form a large pore upon prolonged or repeated agonist stimulation. It is partially activated by saturating concentrations of ATP, whereas it is fully activated by the synthetic ATP analog benzoylbenzoic ATP (BzATP) (Bianchi et al., Eur. J. Pharmacol. Vol. 376, pages 127-138, 1999). The P2X₇ receptor is expressed by presynaptic terminals in the central and peripheral nervous systems, antigen-presenting cells including macrophages, human epidermal Langerhans' cells, microglial cells and a number of tumor cell lines of varying origin (Jacobson K A, et al. “Adenosine and Adenine Nucleotides: From Molecular Biology to Integrative Physiology”. L. Belardinelli and A. Pelleg (eds.), Kluwer, Boston, pages 149-166, 1995).

Recent studies demonstrated the participation of P2X₇ receptors in the modulation of electrical stimulation and ATP-evoked GABA and glutamate release from mouse hippocampal slices (Papp et al., Neuropharmacology and Neurotoxicology Vol. 15, pages 2387-2391, 2004)). In the central nervous system, the P2X₇ receptor is predominately expressed by microglia, the resident macrophages of the brain. On glial cells, the P2X₇ receptor has been shown to mediate release of glutamate (Anderson C. et al. Drug Dev. Res. Vol. 50, page 92, 2000). Upregulation of the P2X₇ receptor, most likely on activated microglia, was reported in association with ischemic damage and necrosis induced by occlusion of middle cerebral artery in rat brain (Collo G. et al. Neuropharmacology Vol. 36, pages 1277-1283, 1997). Recent studies indicate a role of the P2X₇ receptor in the generation of superoxide in microglia, and upregulation of P2X₇ receptors around β-amyloid plaques in a transgenic mouse model for Alzheimer's disease (Parvathenani et al., J. Biol. Chemistry. Vol. 278, pages 13300-13317, 2003) and in multiple sclerosis lesions from autopsy brain sections (Narcisse et al., Glia. Vol. 49, pages 245-258 (2005).

Activation of the P2X₇ receptor on cells of the immune system (macrophages, mast cells and lymphocytes) leads to release of interleukin-1β (IL-1β), giant cell formation, degranulation, and L-selection shedding. ATP has been shown to increase local release and process of IL-1β following lipopolysaccharide S (LPS) intraperitoneal injections in rats through a P2X₇ receptor mediated mechanism (Griffiths et al., J. Immunology Vol. 154, pages 2821-2828 (1995), Solle et al., J. Biol. Chemistry. Vol. 276, pages 125-132, (2001)).

Oxidized ATP (oATP), a nonselective and irreversible P2X₇ antagonist, was recently reported to possess peripherally mediated antinociceptive properties in inflamed rats (Dell' Antonio et al. Neuroscience Lett., Vol. 327, pages 87-90, (2002)). Activation of P2X₇ receptors localized on presynaptic terminals in the central and peripheral nervous systems (Deuchars et al J. Neuroscience. Vol. 21, pages 7143-7152, (2001)) induced release of the excitatory amino acid neurotransmitter glutamate.

Studies from mice lacking P2X₇ receptor resulted in absence of inflammatory and neuropathic hypersensitivity to mechanical and thermal stimuli, indicating a link between a P2X₇ purinoceptor gene and inflammatory and neuropathic pain (Chessell et al., Pain, Vol 114, pages 386-396, (2005)).

Studies with P2X₇ receptor-deficient mice showed a decreased susceptibility to monoclonal anti-collagen-induced arthritis, a model for inflammatory joint disease, indicating the involvement of P2X₇ receptor activation in proinflammatory mechanisms (Labasi et al., J. of Immunology Vol 168, pages 6436-6445, (2002))

Antagonists to the P2X₇ receptor significantly improved functional recovery and decreased cell death in spinal cord injury (SCI) animal models. Rats with SCI were administered P2X₇ receptor irreversible antagonists oATP and PPADS with a resulting decrease of histological injury and improved recovery of motor function after the lesions (Wang et al., Nature Medicine Vol. 10, pages B21-B27, (2004)).

Taken together, these findings indicate that compounds that are ligands of the P2X₇ receptor may have utility in the treatment of pain, inflammatory processes, and degenerative conditions associated with disease states such as rheumatoid arthritis, osteoarthritis, psoriasis, allergic dermatitis, asthma, chronic obstructive pulmonary disease, airways hyper-responsiveness, septic shock, glomerulonephritis, irritable bowel disease, Crohn's disease, ulcerative colitis, atherosclerosis, growth and metastases of malignant cells, myoblastic leukaemia, diabetes, Alzheimer's disease, multiple sclerosis, meningitis, osteoporosis, burn injury, ischemic heart disease, stroke and varicose veins.

In view of the above facts, there is a need for compounds that are selective P2X₇ antagonist that can be efficiently used in preventing, treating, or ameliorating states as neuropathic pain, chronic inflammatory pain, inflammation, rheumatoid arthritis, depression and neurodegenerative conditions associated with several progressive CNS disorders, including, but not limited to, Alzheimer's disease, Parkinson's disease, depression, amyotrophic lateral sclerosis, Huntington's disease, dementia with Lewy bodies, multiple sclerosis as well as diminished CNS function resulting from traumatic brain injury.

### SUMMARY OF THE INVENTION

The invention is directed to selective P2X₇ antagonist compounds as well as compositions comprising such compounds, and method of using the same. Compounds of the invention have the formula:

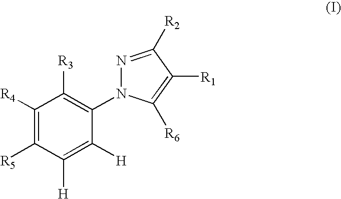

or a pharmaceutically acceptable salt, prodrug, salt of prodrug, or a combination thereof, wherein

R₁ is hydrogen or —CN, and R₂ is hydrogen, or

R₁ and R₂ together with the carbon atoms to which they are attached, form a monocyclic saturated ring consisting of 5, 6 or 7 carbon atoms and one of the carbon atoms of the ring is optionally replaced by a heteroatom selected from the group consisting of S, N, NH, O, SO and SO₂; and said ring is optionally substituted with 1 or 2 substituents selected from the group consisting of alkyl, halogen, haloalkyl, —C(O)alkyl, and —S(O)₂alkyl;

R₃ is halogen, —CN, haloalkyl, alkoxy or haloalkoxy;

R₄ is alkyl, halogen, —CN, haloalkyl, alkoxy or haloalkoxy;

R₅ is hydrogen, alkyl, halogen, —CN, haloalkyl, alkoxy or haloalkoxy;

R₆ is —N(H)—W, or —N(H)—C(Rₓ)(H)—W₁; wherein
Rₓ is hydrogen, alkyl or haloalkyl,
W is 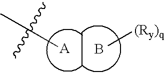
wherein
A is a five or six membered monocyclic ring selected from the group consisting of cycloalkyl and heterocycle and is optionally substituted with 1, 2, or 3 substituents selected from the group consisting of alkyl, halo and haloalkyl;
B is phenyl or monocyclic heteroaryl, optionally substituted with 1, 2 or 3 substituents selected from the group consisting of halo, alkyl, —CN, —OR_{A}, —SR_{A}, —N(R_{A})(R_{B}) and haloalkyl;
q is 0 or 1;
R_{y} is X or -L-X;

W₁ is phenyl or monocyclic heteroaryl, wherein each W₁ is optionally fused with a monocyclic, five or six-membered ring selected from the group consisting of phenyl, heteroaryl, heterocycle, cycloalkyl and cycloalkenyl; wherein each ring as represented by W₁ is independently unsubstituted, substituted with one, two or three R₇, or substituted with zero, one or two R₇ and one substituent selected from the group consisting of X and -L-X;

L at each occurrence is independently O, N(H), N(alkyl), S, S(O), S(O)₂, S(O)₂N(H), SO₂N(alkyl), N(H)S(O)₂, N(alkyl)S(O)₂, CON(H), CON(alkyl), N(H)CO, or N(alkyl)CO);

X, at each occurrence is independently aryl, heteroaryl, cycloalkyl, cycloalkenyl, or heterocycle; each of which is independently unsubstituted or substituted with one, two or three R₇;

R₇ at each occurrence is independently alkyl, alkenyl, CN, NO₂, halo, ═O, —OR_{A}, —SR_{A}, —S(O)R_{A}, —S(O)₂R_{A}, —S(O)₂N(R_{A})(R_{B}), —N(R_{A})(R_{B}), —C(O)R_{A}, —C(O)OR_{A}, —C(O)N(R_{A})(R_{B}), haloalkyl, -alkyl-OR_{A}, -alkyl-SR_{A}, -alkyl-S(O)R_{A}, -alkyl-S(O)₂R_{A}, -alkyl-S(O)₂N(R_{A})(R_{B}), -alkyl-N(R_{A})(R_{B}), -alkyl-C(O)R_{A}, -alkyl-C(O)OR_{A}, or -alkyl-C(O)N(R_{A})(R_{B});

R_{A} at each occurrence is independently hydrogen, alkyl, alkenyl or haloalkyl; and

R_{B} at each occurrence is independently hydrogen, alkyl, or haloalkyl.

### DETAILED DESCRIPTION OF THE INVENTION

All references contained herein are fully incorporated by reference.

a) Definition of Terms

The term “alkoxy” as used herein, means an alkyl group, as defined herein, appended to the parent molecular moiety through an oxygen atom. Representative examples of alkoxy include, but are not limited to, methoxy, ethoxy, propoxy, isopropoxy, butoxy, isobutoxy, tert-butoxy, pentyloxy, and hexyloxy.

The term “alkyl” as used herein, means a straight or branched chain hydrocarbon containing from 1 to 10 carbon atoms. Representative examples of alkyl include, but are not limited to, methyl, ethyl, n-propyl, isopropyl, n-butyl, sec-butyl, isobutyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “aryl” as used herein, means phenyl or a bicyclic aryl. The bicyclic aryl is naphthalenyl, or a phenyl fused to a monocyclic cycloalkyl, or a phenyl fused to a monocyclic cycloalkenyl. The phenyl and the bicyclic aryl groups of the present invention are unsubstituted or substituted. The bicyclic aryl is attached to the parent molecular moiety through any carbon atom contained within the bicyclic aryl. Representative examples of the aryl groups include, but are not limited to, dihydroindenyl, 2,3-dihydro-1H-inden-1-yl, naphthalenyl, dihydronaphthalenyl, and 1,2,3,4-tetrahydronaphthalen-1-yl.

The term “cycloalkyl” or “cycloalkane” as used herein, means a monocyclic or bicyclic cycloalkyl. The monocyclic cycloalkyl is a hydrocarbon ring consisting of three to eight carbon atoms, zero heteroatom and single carbon-carbon bonds within the ring. The monocyclic cycloalkyl can be attached to the parent molecular moiety through any substitutable atom contained within the monocyclic cycloalkyl. Examples of monocyclic cycloalkyls include, but are not limited to, cyclopropyl, cyclobutyl, cyclopentyl, cyclohexyl, cycloheptyl, and cyclooctyl. The bicyclic cycloalkyl is a monocyclic cycloalkyl fused to a monocyclic cycloalkyl. The bicyclic cycloalkyl can be attached to the parent molecular moiety through any substitutable atom contained within the bicyclic cycloalkyl. The monocyclic and bicyclic cycloalkyl groups of the present invention can be unsubstituted or substituted

The term “cycloalkenyl” or “cycloalkene” as used herein, means a monocyclic or a bicyclic hydrocarbon ring system. The monocyclic cycloalkenyl has four-, five-, six-, seven- or eight carbon atoms and zero heteroatom within the ring. The four-membered ring systems have one double bond, the five- or six-membered ring systems have one or two double bonds, and the seven- or eight-membered ring systems have one, two or three double bonds. The monocyclic cycloalkenyl can be attached to the parent molecular moiety through any substitutable atom contained within the monocyclic cycloalkenyl. Representative examples of mocyclic cycloalkenyl groups include, but are not limited to, cyclobutenyl, cyclopentenyl, cyclohexenyl, cycloheptenyl and cyclooctenyl. The bicyclic cycloalkenyl is a monocyclic cycloalkenyl fused to a monocyclic cycloalkyl group, or a monocyclic cycloalkenyl fused to a monocyclic cycloalkenyl group. The bicyclic cycloalkenyl can be attached to the parent molecular moiety through any substitutable atom contained within the bicyclic cycloalkenyl. Representative examples of the bicyclic cycloalkenyl groups include, but are not limited to, 4,5,6,7-tetrahydro-3aH-indene, octahydronaphthalenyl and 1,6-dihydro-pentalene. The monocyclic and bicyclic cycloalkenyl groups of the present invention can be unsubstituted or substituted.

The term “halo” or “halogen” as used herein, means —Cl, —Br, —I or —F.

The term “haloalkoxy” as used herein, means an alkoxy group, as defined herein, in which one, two, three, four, five or six hydrogen atoms are replaced by halogen. Representative examples of haloalkoxy include, but are not limited to, chloromethoxy, 2-fluoroethoxy, trifluoromethoxy, 2-chloro-3-fluoropentyloxy, and pentafluoroethoxy.

The term “haloalkyl” as used herein, means an alkyl group, as defined herein, in which one, two, three, four, five or six hydrogen atoms are replaced by halogen. Representative examples of haloalkyl include, but are not limited to, chloromethyl, 2-fluoroethyl, trifluoromethyl, difluoromethyl, pentafluoroethyl, and 2-chloro-3-fluoropentyl and trifluoroethyl.

The term “heterocycle” or “heterocyclic” as used herein, means a monocyclic heterocycle or a bicyclic heterocycle. The monocyclic heterocycle is a three-, four-, five-, six- or seven-membered ring containing at least one heteroatom independently selected from the group consisting of O, N, N(H) and S. The three- or four-membered ring contains zero or one double bond and a heteroatom selected from the group consisting of O, N, N(H) and S. The five-membered ring contains zero or one double bond, and one, two or three heteroatoms selected from the group consisting of O, N, N(H) and S. The six-membered ring contains zero, one or two double bonds and one, two or three heteroatoms selected from the group consisting of O, N, N(H) and S. The seven-membered ring contains zero, one, two, or three double bonds and one, two or three heteroatoms selected from the group consisting of O, N, N(H) and S. The monocyclic heterocycle can be unsubstituted or substituted and is connected to the parent molecular moiety through any substitutable carbon atom or any substitutable nitrogen atom contained within the monocyclic heterocycle. Representative examples of monocyclic heterocycle include, but are not limited to, azetidinyl, azepanyl, aziridinyl, diazepanyl, [1,4]diazepan-1-yl, 1,3-dioxanyl, 1,3-dioxolanyl, 1,3-dithiolanyl, 1,3-dithianyl, homomorpholinyl, homopiperazinyl, imidazolinyl, imidazolidinyl, isothiazolinyl, isothiazolidinyl, isoxazolinyl, isoxazolidinyl, morpholinyl, oxadiazolinyl, oxadiazolidinyl, oxazohnyl, oxazolidinyl, piperazinyl, piperidinyl, pyranyl, pyrazolinyl, pyrazolidinyl, pyrrolinyl, pyrrolidinyl, tetrahydrofuranyl, tetrahydropyranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to a phenyl group, or a monocyclic heterocycle fused to a monocyclic cycloalkyl, or a monocyclic heterocycle fused to a monocyclic cycloalkenyl, a monocyclic heterocycle fused to a monocyclic heterocycle, or a monocyclic heterocycle fused to a monocyclic heteroaryl. The bicyclic heterocycle is connected to the parent molecular moiety through any substitutable carbon atom or any substitutable nitrogen atom contained within the bicyclic heterocycle and can be unsubstituted or substituted. Representative examples of bicyclic heterocycle include, but are not limited to, benzodioxinyl, benzopyranyl, thiochromanyl, 2,3-dihydroindolyl, indolizinyl, pyranopyridinyl, 1,2,3,4-tetrahydroisoquinolinyl, 1,2,3,4-tetrahydroquinolinyl, thiopyranopyridinyl, 2-oxo-1,3-benzoxazolyl, 3-oxo-benzoxazinyl, 3-azabicyclo[3.2.0]heptyl, 3,6-diazabicyclo[3.2.0]heptyl, octahydrocyclopenta[c]pyrrolyl, hexahydro-1H-furo[3,4-c]pyrrolyl, octahydropyrrolo[3,4-c]pyrrolyl, 2,3-dihydrobenzofuran-7-yl, 2,3-dihydrobenzofuran-3-yl, and 3,4-dihydro-2H-chromen-4-yl. The monocyclic or bicyclic heterocycles as defined herein may have two of the non-adjacent carbon atoms connected by a heteroatom selected from N, N(H), O or S, or an alkylene bridge of between one and three additional carbon atoms. Representative examples of monocyclic or bicyclic heterocycles that contain such connection between two non-adjacent carbon atoms include, but not limited to, 2-azabicyclo[2.2.2]octyl, 2-oxa-5-azabicyclo[2.2.2]octyl, 2,5-diazabicyclo[2.2.2]octyl, 2-azabicyclo[2.2.1]heptyl, 2-oxa-5-azabicyclo[2.2.1]heptyl, 2,5-diazabicyclo[2.2.1]heptyl, 2-azabicyclo[2.1.1]hexyl, 5-azabicyclo[2.1.1]hexyl, 3-azabicyclo[3.1.1]heptyl, 6-oxa-3-azabicyclo[3.1.1]heptyl, 8-azabicyclo[3.2.1]octyl, 3-oxa-8-azabicyclo[3.2.1]octyl, 1,4-diazabicyclo[3.2.2]nonyl, 1,4-diazatricyclo[4.3.1.1^{3,8}]undecyl, 3,10-diazabicyclo[4.3.1]decyl, or 8-oxa-3-azabicyclo[3.2.1]octyl, octahydro-1H-4,7-methanoisoindolyl, and octahydro-1H-4,7-epoxyisoindolyl. The nitrogen heteroatom may or may not be quaternized, and may or may not be oxidized to the N-oxide. In addition, the nitrogen containing heterocyclic rings may or may not be N-protected.

The term “heteroaryl” as used herein, means a monocyclic heteroaryl or a bicyclic heteroaryl. The monocyclic heteroaryl is a five- or six-membered ring. The five-membered ring consists of two double bonds and one sulfur, nitrogen or oxygen atom. Alternatively, the five-membered ring has two double bonds and one, two, three or four nitrogen atoms and optionally one additional heteroatom selected from oxygen or sulfur, and the others carbon atoms. The six-membered ring consists of three double bonds, one, two, three or four nitrogen atoms, and the others carbon atoms. The bicyclic heteroaryl consists of a monocyclic heteroaryl fused to a phenyl, or a monocyclic heteroaryl fused to a monocyclic cycloalkyl, or a monocyclic heteroaryl fused to a monocyclic cycloalkenyl, or a monocyclic heteroaryl fused to a monocyclic heteroaryl. The monocyclic and the bicyclic heteroaryl are connected to the parent molecular moiety through any substitutable atom contained within the monocyclic or the bicyclic heteroaryl. The monocyclic and bicyclic heteroaryl groups of the present invention can be substituted or unsubstituted. In addition, the nitrogen heteroatom may or may not be quaternized, and may or may not be oxidized to the N-oxide. Also, the nitrogen containing rings may or may not be N-protected. Representative examples of monocyclic heteroaryl include, but are not limited to, furanyl, imidazolyl, isoxazolyl, isothiazolyl, oxadiazolyl, oxazolyl, pyridinyl, pyridine-N-oxide, pyridazinyl, pyrimnidinyl, pyrazinyl, pyrazolyl, pyrrolyl, tetrazolyl, thiadiazolyl, thiazolyl, thienyl, triazolyl, and triazinyl. Representative examples of bicyclic heteroaryl groups include, but not limited to, benzothienyl, benzoxazolyl, benzimidazolyl, benzoxadiazolyl, 6,7-dihydro-1,3-benzothiazolyl, imidazo[1,2-a]pyridinyl, indazolyl, 1H-indazol-3-yl, indolyl, isoindolyl, isoquinolinyl, naphthyridinyl, pyridoimidazolyl, quinolinyl, quinolin-8-yl, and 5,6,7,8-tetrahydroquinolin-5-yl.

b) Compounds, Methods and Compositions of the Invention

Compounds of the invention have the formula (I) as described above. More particularly, compounds of formula (I) can include, but are not limited to, compounds wherein R₁ is hydrogen or —N, R₂ is hydrogen; and R₆ is —N(H)—C(Rₓ)(H)—W₁. Preferred compounds are those in which Rₓ is hydrogen and W₁ is phenyl or monocyclic heteroaryl independently unsubstituted, substituted with one, two or three R₇, or substituted with zero, one or two R₇ and one substituent selected from the group consisting of X and -L-X. More preferred compounds include those in which R₁ is hydrogen; W₁ is substituted phenyl; R₃ and R₄ are halogen; R₅ is hydrogen and, R₇ is alkyl.

Other compounds of the present invention include those in which R₁ is —CN; R₃ and R₄ are halogen; R₅ is hydrogen and W₁ is unsubstituted phenyl; also included are those compounds in which R₁ is —CN; R₃ and R₄ are halogen; R₅ is hydrogen; and W₁ is unsubstituted monocyclic heteroaryl. The present invention also includes compounds wherein R₁ is —CN; R₃ and R₄ are halogen; W₁ is monocyclic heteroaryl substituted with R₇ and R₇ is alkyl.

Other compounds of the present invention are those in which R₁ and R₂ together with the carbon atoms to which they are attached, form a monocyclic saturated ring consisting of 5, 6 or 7 carbon atoms and one of the carbon atoms of the ring is optionally replaced by a heteroatom selected from the group consisting of S, N, NH, O, SO and SO₂; and said ring is optionally substituted with 1 or 2 substituents selected from the group consisting of alkyl, halogen, haloalkyl, —C(O)alkyl, and —S(O)₂alkyl; and

R₆ is —N(H)—C(Rₓ)(H)—W₁. Preferred compounds include those where R₁ and R₂ together with the carbon atoms to which they are attached, form a 5 carbon monocyclic saturated ring; Rₓ is hydrogen; R₃ is halogen; R₄ is halogen; and W₁ is phenyl or monocyclic heteroaryl. Most preferred compounds are those in which W₁ is phenyl substituted with R₇ and R₇ is alkyl. Other preferred compounds include those wherein W₁ is phenyl substituted with L-X, wherein L is O, and X is aryl, preferably phenyl Other preferred compounds are those in which W₁ is unsubstituted monocyclic heteroaryl. Other compounds include those wherein W₁ is monocyclic heteroaryl substituted with R₇ and R₇ is preferably alkyl, or those in which W₁ is monocyclic heteroaryl substituted with L-X, wherein L is O, and X is aryl.

The present invention also comprises compounds of formula (I) as described above, in which R₁ and R₂ together with the carbon atoms to which they are attached, form a 5 carbon monocyclic saturated ring and one of the carbon atoms of the ring is replaced by S, SO or SO₂; preferably S. Preferred compounds are those in which R₃ is halogen; R₄ is halogen; R₆ is —N(H)—C(Rₓ)(H)—WI; Rₓ is hydrogen; and W₁ is phenyl or monocyclic heteroaryl, most preferably phenyl substituted with R₇, and R₇ is alkyl or monocyclic heteroaryl substituted with R₇ and R₇ is alkyl.

Other preferred compounds include those where W₁ is monocyclic heteroaryl substituted with L-X, wherein L is O, and X is aryl. The present invention also includes compounds of formula (I) wherein R₁ and R₂ together with the carbon atoms to which they are attached, form a 6 carbon monocyclic saturated ring; R₃ is halogen; R₄ is halogen; R₆ is —N(H)—C(Rₓ)(H)—W₁; Rₓ is hydrogen; and W₁ is phenyl or monocyclic heteroaryl. Preferred compounds include those wherein W₁ is monocyclic heteroaryl substituted with R₇, and R₇ is alkyl.

The present invention also includes compounds of formula (I) wherein R₁ is hydrogen or —CN; R₂ is hydrogen; R₆ is —N(H)—W, wherein

W is 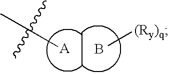
A can be a five or six membered monocyclic ring selected from the group consisting of cycloalkyl and heterocycle and is optionally substituted with 1, 2, or 3 substituents selected from the group consisting of alkyl, halo and haloalkyl; B is phenyl or monocyclic heteroaryl, optionally substituted with 1, 2 or 3 substituents selected from the group consisting of halo, alkyl, —CN, —OR_{A}, —SR_{A}, —N(R_{A})(R_{B}) and haloalkyl; q is 0 or 1; and R_{y} is X or -L-X. Other compounds of the present invention are those compounds of formula (I) wherein R₁ and R₂ together with the carbon atoms to which they are attached, form a monocyclic saturated ring consisting of 5, 6 or 7 carbon atoms and one of the carbon atoms of the ring is optionally replaced by a heteroatom selected from the group consisting of S, N, NH, O, SO and SO₂; R₆ is —N(H)—W, wherein

W is 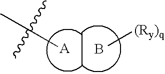
and A is a five or six membered monocyclic ring selected from the group consisting of cycloalkyl and heterocycle and is optionally substituted with 1, 2, or 3 substituents selected from the group consisting of alkyl, halo and haloalkyl; B is phenyl or monocyclic heteroaryl, optionally substituted with 1, 2 or 3 substituents selected from the group consisting of halo, alkyl, —CN, —OR_{A}, —SR_{A}, —N(R_{A})(R_{B}) and haloalkyl; q is 0 or 1; and R_{y} is X or -L-X. The invention also provides pharmaceutical compositions comprising a therapeutically effective amount of one or more of the compounds of formula (I) in combination with a pharmaceutically acceptable carrier. The compositions comprise compounds of the invention formulated together with one or more non-toxic pharmaceutically acceptable carriers. The pharmaceutical compositions can be formulated for oral administration in solid or liquid form, for parenteral injection or for rectal administration.

The term “pharmaceutically acceptable carrier,” as used herein, means a non-toxic, inert solid, semi-solid or liquid filler, diluents, encapsulating material or formulation auxiliary of any type. Some examples of materials which can serve as pharmaceutically acceptable carriers are sugars, cellulose and its derivatives, powdered tragacanth, malt, gelatin, talc, cocoa butter and suppository waxes, oils, glycols, esters, agar, buffering agents such as magnesium hydroxide and aluminum hydroxide, alginic acid, pyrogen-free water, isotonic saline, Ringer's solution, ethyl alcohol, and phosphate buffer solutions, as well as other non-toxic compatible lubricants, as well as coloring agents, releasing agents, coating agents, sweetening, flavoring and perfuming agents. Preservatives and antioxidants can also be present in the composition, according to the judgment of one skilled in the art of formulations.

The compounds of the invention can be used in the form of pharmaceutically acceptable salts. The term “pharmaceutically acceptable salt” refers to salts of compounds of formula (I) which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well-known in the art.

The term “pharmaceutically acceptable prodrug” or “prodrug,” as used herein, represents those prodrugs of the compounds of the invention which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the invention can be rapidly transformed in vivo to a parent compound of formula (I), for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

The invention contemplates pharmaceutically active compounds either chemically synthesized or formed by in vivo biotransformation to compounds of formula (I).

Compounds and compositions of the invention are useful for modulating the effects of P2X₇ receptors. In particular, the compounds and compositions of the invention can be used for treating and preventing disorders modulated by P2X₇ receptors. Typically, such disorders can be ameliorated by selectively modulating the P2X₇ receptors in a mammal, preferably by administering a compound or composition of the invention, either alone or in combination with another active agent, for example, as part of a therapeutic regimen.

The compounds of the invention, including but not limited to those specified in the examples, possess an affinity for P2X₇ receptors and display antagonist activity at the P2X₇ receptors. Therefore, the compounds of the invention can be useful for the treatment and prevention of a number of P2X₇ receptor-mediated diseases or conditions.

For example, P2X₇ receptors have been shown to play a significant role in ischemic damage and necrosis induced by occlusion of middle cerebral artery in rat brain (Collo G. et al. Neuropharmacology Vol. 36, pages 1277-1283, 1997), therefore the compounds of the present invention, that are P2X₇ receptor antagonists may be useful in preventing or treating neurodegenerative diseases. P2X₇ receptors are also involved in the formation of β-amyloid plaques in a transgenic mouse model for Alzheimer's disease (Parvathenani et al., J. Biol. Chemistry. Vol. 278, pages 13300-13317, 2003), and in multiple sclerosis lesions as shown in autopsy brain sections (Narcisse et al., Glia. Vol. 49, pages 245-258 (2005). Therefore, the compounds of the present invention, that are P2X₇ receptor antagonists may be useful in preventing or treating neurodegenerative conditions associated with several progressive CNS disorders, including, but not limited to, Alzheimer's disease, Parkinson's disease, depression, amyotrophic lateral sclerosis, Huntington's disease, dementia with Lewy bodies, multiple sclerosis as well as diminished CNS function resulting from traumatic brain injury.

Blockade of P2X₇ receptor with oxidized ATP (a nonselective and irreversible antagonist) induces peripherally mediated antinociceptive properties in inflamed rats (Dell' Antonio et al. Neuroscience Lett. Vol. 327, pages 87-90, (2002)). Similarly, studies with mice lacking P2X₇ receptor indicate a link between a P2X₇ purinoceptor gene and inflammatory and neuropathic pain (Chessell et al., Pain, Vol 114, pages 386-396, (2005)), and the involvement of P2X₇ receptor activation in proinflammatory mechanisms (Labasi et al., J. of Immunology Vol 168, pages 6436-6445, (2002)). Thus, the compounds of the present invention by being P2X₇ receptor antagonists can be useful for preventing or treating states as neuropathic pain, chronic inflammatory pain, inflammation, and rheumatoid arthritis.

Antagonists to the P2X₇ receptor significantly improved functional recovery and decreased cell death in spinal cord injury (SCI) animal models. Rats with SCI were administered P2X₇ receptor irreversible antagonists oATP and PPADS with a resulting decrease of histological injury and improved recovery of motor function after the lesions (Wang et al., Nature Medicine Vol. 10, pages B21-B27, (2004)).

In view of these results, the P2X₇ receptor antagonists of the present invention can prove useful to preventing, treating, or ameliorating states as depression, neurodegenerative conditions associated with several progressive CNS disorders, including, but not limited to, Alzheimer's disease, Parkinson's disease, depression, amyotrophic lateral sclerosis, Huntington's disease, dementia with Lewy bodies, multiple sclerosis as well as diminished CNS function resulting from traumatic brain injury.

c) Preparation of the Compounds of the Invention

The compounds and processes of the present invention will be better understood in connection with the following synthetic schemes, which illustrate the methods by which the compounds of the invention may be prepared. Starting materials can be obtained from commercial sources or prepared by well-established literature methods known to those of ordinary skill in the art.

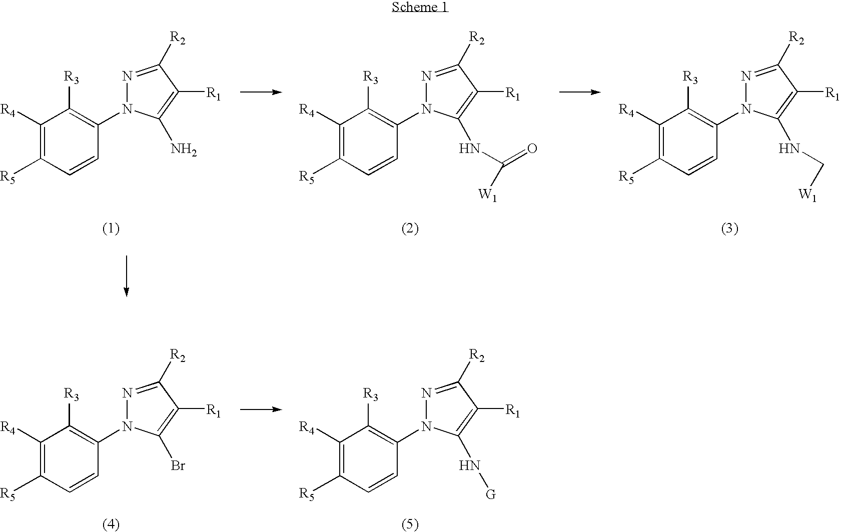

Compounds of formula (3) wherein R₁, R₂, R₃, R₄, R₅ and W₁ are as defined in formula (I) can be prepared from aminopyrazoles of formula (I) as shown in Scheme 1.

Aminopyrazoles of formula (I), purchased or prepared using known methodologies, when treated with substituted carboxylic acids of formula W₁COOH, in the presence of a coupling reagent such as, but not limited to, O-(7-azabenzotriazol-1-yl)-N,N,N′,N′-tetramethyluronium hexafluorophosphate (HATU) and a base such as, but not limited to triethylamine or diisopropyl ethyl amine, provide amides of formula (2). The reaction is generally conducted in a solvent such as, but not limited to, N,N-dimethylformamide, dichloromethane, tetrahydrofluan, or mixture thereof, at about room temperature. Alternatively, the amides of formula (2) can be prepared by reacting aminopyrazoles of formula (I) with acid chlorides of formula W₁COCl (purchased or prepared from the corresponding acids with thionyl chloride using known methodologies), in the presence of a base such as, but not limited to, triethylamine or pyridine. The reaction is generally conducted at a temperature from about 0° C. to about 50° C. and in a solvent such as, but not limited to, dichloromethane, tetrahydrofuran, or diethylether Amides of formula (2) can be treated with a reducing agent such as, but not limited to, borane, lithium aluminum hydride, aluminum hydride, to provide compounds of formula (3).

Aminopyrazoles of formula (1) can also be converted to compounds of formula (5) wherein G is W or —C(Rₓ)(H)—W, and W, W₁, R₁, R₂, R₃, R₄, and R₅ are as defined in formula (I). Aminopyrazoles of formula (1) can be transformed to the corresponding bromides of formula (4) by treatment with bromoform and t-butylnitrile at elevated temperature. Bromides of formula (4) when treated with amines of formula G-NH₂ at a temperature from about 100° C. to about 200° C., affords compounds of formula (5). The reaction can be performed neat or in a solvent such as, but not limited to, toluene, xylenes, dichlorobenzene, dioxane, diphenylether, or N,N-dimethylformamide.

Amines of formula G-NH₂ are either commercially available or can be prepared using known methodologies. Literature references that outline synthesis of amines of formula G-NH₂ include, but are not limited to, Kaluza et al Chem. Ber. 1955, 88, 597; Bennett et al J. Chem. Soc. 1931; 1692, Sagorewskii et al J. Gen. Chem. USSR 1964; 34, 2294, Pratap et al Indian J. Chem. Sect. B 1981; 20; 1063, WO2005/42533, p121; Braun et al Chem. Ber. 1929, 62, 2420; Tikk et al Acta Chim. Hung. 1986, 121, 255; and Bernabeu et al Synth. Commun. 2004, 34, 137.

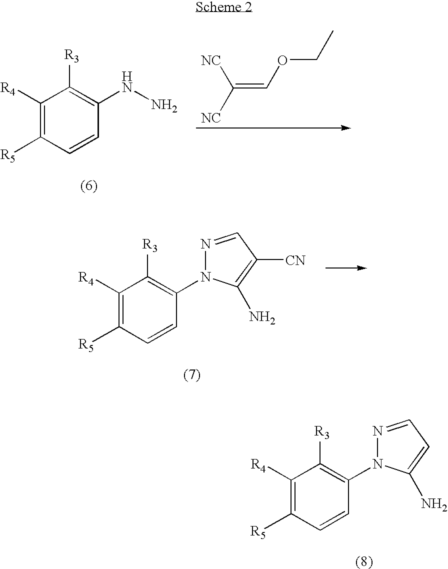

Aminopyrazoles of formula (7) and (8) wherein R₃, R₄ and R₅ are as defined in formula (I) can be prepared using the chemistry outlined in Scheme 2. The hydrochloride salt of substituted arylhydrazines of formula (6) can be reacted with ethoxyethylenemalononitrile in the presence of a base such as sodium hydroxide or sodium ethoxide to provide compounds of formula (7). The reaction is generally carried out in a solvent such as ethanol, and at the reflux temperature of the solvent used. The aminopyrazoles of formula (7) when refluxed with concentrated hydrochloric acid afford compounds of formula (8).

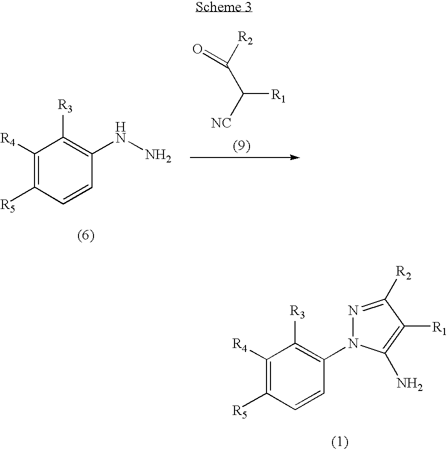

Aminopyrazoles of formula (1) wherein R₃, R₄ and R₅ are as defined in formula (I) and R₁ and R₂ are hydrogen or R₁ and R₂ together with the carbon atoms to which they are attached form a ring as defined in formula (I), can be prepared using the chemistry as outlined in Scheme 3. The hydrochloride salt of substituted arylhydrazines of formula (6) can be reacted with appropriately substituted cyanoketones of formula (9), in a solvent such as ethanol and the like, at the reflux temperature of the solvent employed to provide aminopyrazoles of formula (1).

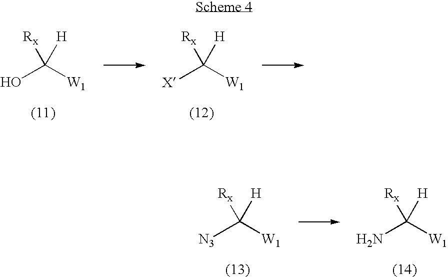

Amines of formula (14) wherein W₁ and Rₓ are as defined in formula (I), can be prepared by a variety of methods known to one skilled in the art. One example of such preparations is outlined in Scheme 4. Alcohols of formula (11) can be reacted with neat thionyl chloride, with or without a solvent at about room temperature to provide chlorides of formula (12) wherein X′ is Cl. Examples of solvents used are, but not limited to, dichloromethane and chloroform. Alternatively, compounds of formula (12) wherein X′ is methyl sulfonate (mesylate) can be prepared from alcohols of formula (11) with methane sulfonyl chloride, in the presence of a base such as, but not limited to, triethylamine.

Displacement of chlorides or mesylates of formula (12) with sodium azide in a solvent such as, but not limited to, N,N-dimethylformamide or acetone, provides azides of formula (13), which can be reduced to amines of formula (14) in the presence of a reducing agent such as, but not limited to, palladium/carbon or PtO₂/carbon. The reaction can be performed in a solvent such as, but not limited to, ethanol, methanol or ethyl acetate at about room temperature.

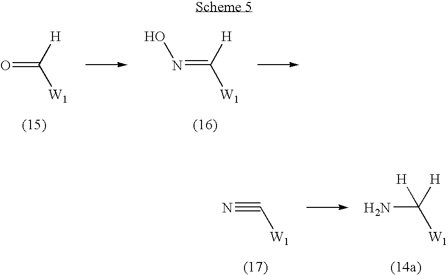

Amines of formula (14a) wherein W₁ is as defined in formula (I) can be prepared from the corresponding aldehydes of formula (15) as shown in Scheme 5. Reaction of the aldehydes of formula (15) with hydroxylarine hydrochloride in an alcoholic solvent such as, but not limited to, ethanol, provides oximes of formula (16). Oximes of formula (16) can be converted to nitrites of formula (17) in the presence of acetic anhydride and a base such as, but not limited to, potassium hydroxide or sodium hydroxide. Reduction of the nitrites of formula (17) with Raney/nickel and ammonia provides amines of formula (14a). The reduction can be performed in an alcoholic solvent such as, but not limited to, methanol.

Certain nitrites of formula (17) can be purchased (for example 2-amino nicotinonitrile) or prepared using procedures described in literature reference such as, but not limited to, Almed et al, Indian Chem. Soc., 1996, 73, 141.

Nitriles of formula (17) can also be prepared from the reaction of the corresponding bromides with zinc cyamide in the presence of a palladium catalyst, such as but not limited to, bis(triphenylphospine)palladium (II) chloride and in a solvent such as N,N-dimethylformamide.

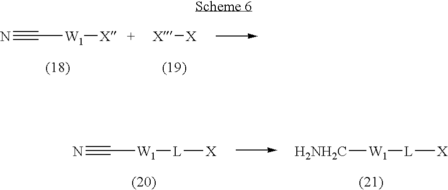

Compounds of formula (20) wherein L is O, N(H), N(alkyl) or S, and W₁ and X are as defined in formula (I) can be prepared by reaction of nitrites of formula (18) wherein X″ is OH, NH₂, N(H)(alkyl) or SH with halides of formula (19) wherein X′″ is fluoro or chloro, in the presence of a base such as, but not limited to, sodium hydride or potassium carbonate. The reaction can be conducted in a solvent such as tetrahydrofuran, dimethylformamide or dioxane at a temperature from about room temperature to about 150° C.

Conversely, compounds of formula (20) wherein L is O, N(H), N(alkyl) or S, and W₁ and X are as defined in formula (I) can also be prepared by reaction of nitrites (18) wherein X″ is fluoro or chloro and compounds of formula (19) wherein X′″ is —NH₂, —N(H)(alkyl), OH or SH under the abovementioned conditions.

Amines of formula (21) can be prepared from nitrites of formula (20) using the transformation conditions for the conversion of compounds of formula (17) to (14a) as outlined in Scheme 5.

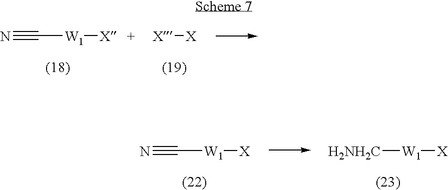

Nitriles of formula (22) wherein W₁ and X are as defined in formula (I), can be prepared by reaction of nitrites of formula (18) wherein X″ is Cl, Br, I or triflate with boronic acid or ester of formula (19) wherein X′″ is —B(OR₁₀₁)₂ and R₁₀₁ is hydrogen or alkyl, in the presence of a palladium catalyst, such as but not limited to, bis(triphenylphospine)palladium (II) chloride and a base such as triethylamine or sodium carbonate. The reaction can be effected by heating from 50-90° C. in solvents such as isopropanol, ethanol, dimethoxyethane, water or dioxane. Alternatively, this transformation can be accomplished by reacting nitrites of formula (18) wherein X″ is Cl, Br, I or triflate with tin reagents of formula (19) wherein X′″ is —Sn(alkyl)₃, with a palladium catalyst such as, but not limited to, tetrakis(triphenylphospine)palladium (0), and cesium fluoride and heating in a solvent such as dioxane. These transformations can also be effected by heating in a microwave reactor.

The transformation can also be accomplished by reacting compounds of formula (19) wherein X′″ is Cl, Br, I or triflate with compounds of formula (18) wherein X″ is —Sn(alkyl)₃ or —B(OR₁₀₁)₂ and R₁₀₁, is hydrogen or alkyl, using the abovementioned reaction conditions.

Amines of formula (23) can be obtained from nitrites of formula (22) using the transformation conditions for the conversion of compounds of formula (17) to (14a) as outlined in Scheme 5.

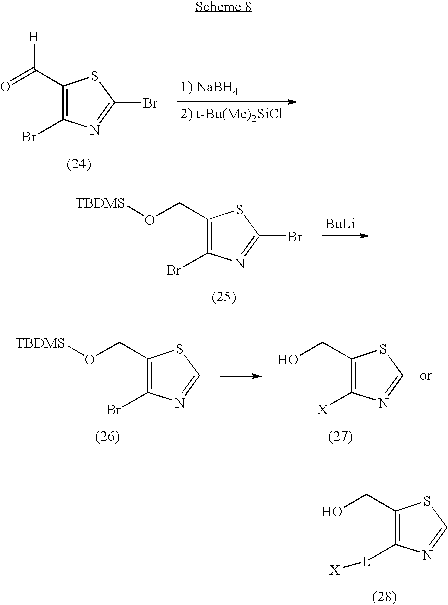

Alcohols of formula (11) can be prepared by various methodologies known to one skilled in the art. One such method is shown in Scheme 8.

The aldehyde of formula (24) can be reduced with sodium borohydride then protected as the t-butyldimethylsilyl ether, followed by mono-debromination with n-butyl lithium to provide mono-bromothiazole of formula (26). The mono-bromothiazole of formula (26) can then be reacted with compounds of formula (19) wherein X′″ is —Sn(alkyl)₃ or —B(OR₁₀₁)₂ and R₁₀₁ is hydrogen or alkyl, using the reaction conditions outlined in Scheme 7, to provide compounds of formula (27) wherein X is as defined in formula (I). The mono-bromothiazole of formula (26) can also be reacted with compounds of formula (19) wherein X′″ is —NH₂, —N(H)(alkyl), or OH, or SH, using the reaction conditions outlined in Scheme 6, to provide compounds of formula (28) wherein L is O, N(H), N(alkyl) or S, and X is as defined as formula (I).

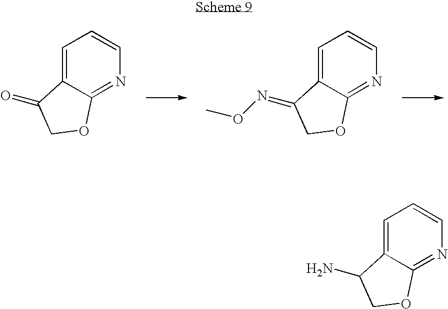

One particular example of amines of formula W—NH₂ can be prepared from its corresponding ketone as shown in Scheme 9.

Furo[2,3-b]pyridin-3(2H)-one (prepared using procedures as described in Morita, Hiroyuki; Shiotani, Shunsaku; J. Heterocycl. Chem.; 23; 1986; 1465-1469) and the hydrochloride salt of methoxylamine can be converted into the methyloxime by reacting at room temperature in a solvent such as pyridine over a period of 24 hours. The intermediate methyloxime, in turn, can be reduced to the corresponding amine by catalytic hydrogenation over Raney Nickel at 4 atmospheres for 6 hours at room temperature in the presence of aqueous ammonia and in an alcoholic solvent such as methanol or ethanol.

d) Reference Examples

The following Examples are intended as an illustration of and not a limitation upon the scope of the invention as defined in the appended claims.

### EXAMPLE 1

### N-[2-(2,3-Dichloro-phenyl)-2H-pyrazol-3-yl]-2-methyl-benzamide

### EXAMPLE 1A

### 5-Amino-1-(2,3-dichloro-phenyl)-1H-pyrazole-4-carbonitrile

(2,3-Dichloro-phenyl)-hydrazine hydrogen chloride (1.0 g, 4.9 mmol) in ethanol (30 mL) was treated with sodium ethoxide (21% in ethanol) (1.8 mL, 4.9 mmol). To the mixture was added 2-ethoxymethylene-malononitrile (570 mg, 4.9 mmol) dropwise. The mixture was refluxed overnight. The reaction mixture was poured into water, and extracted with ethyl acetate (2×). The solvent was removed, and the resulting residue was chromatographed over silica gel eluting with CH₂Cl₂ to give the title compound (850 mg, 69%). MS (DCI/NH₃) m/z 253 (M)⁺.

### EXAMPLE 1B

### 2-(2,3-Dichloro-phenyl)-2H-pyrazol-3-ylamine

The product from Example 1A (850 mg, 3.36 mmol) was treated with concentrated hydrochloric acid (15 mL). The mixture was refluxed for 12 hr, the solvent removed in vacuo, the pH of the mixture was adjusted to 11 with concentrated ammonium hydroxide, and the mixture extracted with ethyl acetate (2×). The solvent was removed in vacuo and the resulting residue was chromatographed over silica gel eluting with CH₂Cl₂ to give the title compound (550 mg, 72%). MS (DCI/NH₃) m/z 228 (M)⁺, 230 (M+2)⁺. ¹H NMR (400 MHz, DMSO-d₆)δ ppm 5.19 (s, 2H) 5.38 (d, J=1.84 Hz, 1H) 7.28 (d, J=1.84 Hz, 1H) 7.42 (dd, J=7.98, 1.84 Hz, 1H) 7.49 (t, J=7.98 Hz, 1H) 7.76 (dd, J=7.98, 1.53 Hz, 1H).

### EXAMPLE 1C

### N-[2-(2,3-Dichloro-phenyl)-2H-pyrazol-3-yl]-2-methyl-benzamide

The product from Example 1B (550 mg, 2.4 mmol) in pyridine (10 mL) was treated dropwise with 2-methylbenzoyl chloride (556 mg, 3.6 mmol) at 0° C. The reaction mixture was allowed to warm to rt overnight, the solvent removed in vacuo, and the resulting residue was chromatographed over silica gel eluting with hexane/ethyl acetate (gradient elution from 6:1 to 4:1) to give the title compound (526 mg, 63%). MS (DCI/NH₃) m/z 346 (M)⁺, 348 (M+2)⁺. ¹HNMR (400 MHz, DMSO-d) δ ppm 2.18 (s, 3H) 6.57 (s, 1H) 7.24 (m, 3H) 7.33 (td, J=7.36, 1.53 Hz, 1H) 7.50 (s, 1H) 7.51 (s, 1H) 7.70 (d, J=1.84 Hz, 1H) 7.79 (t, J=4.60 Hz, 1H) 10.40 (brs, 1H).

### EXAMPLE 2

### [2-(2,3-Dichloro-phenyl)-2H-pyrazol-3-yl]-(2-methyl-benzyl)-amine

The product from Example 1C (280 mg, 0.81 mmol) in tetrahydrofuran (30 mL) was treated with BH₃ (1.0 M in tetrahydrofuran) (4.05 mL, 4.05 mmol) at rt. The reaction mixture was heated at 65° C. overnight, quenched with MeOH, concentrated in vacuo and to the residue was added MeOH (4 mL) and concentrated HCl (1 mL) at rt. The mixture was heated at 60° C. for 6 hr, then stirred at rt overnight. The mixture was adjusted to pH=7 with 5 N NaOH, and extracted with ethyl acetate (2×). The organic layer was concentrated in vacuo and the resulting residue was purified by preparative high pressure liquid chromatography on a Waters Symmetry C18 column (40 mm×100 mm, 7 μm particle size) using a gradient of 10%: 85%: 5% (acetonitrile: H₂O: ammonium acetate) to 95%: 4%: 1% (acetonitrile: H₂O: ammonium acetate) at a flow rate of 40 mL/min. to give the title compound (56.9 mg, 21%). MS (DCI/NH₃) m/z 332 (M)⁺, 334 (M+2)⁺. ¹H NMR (500 MHz, DMSO-d₆) δ ppm 2.26 (s, 3H) 4.14 (d, J=5.80 Hz, 2H) 5.34 (d, J=1.83 Hz, 1H) 5.91 (t, J=5.49 Hz, 1H) 7.13 (m, 3H) 7.26 (t, J=4.58 Hz, 1H) 7.32 (d, J=1.83 Hz, 1H) 7.46 (dd, J=7.93, 1.53 Hz, 1H) 7.51 (t, J=7.93 Hz, 1H) 7.78 (dd, J=7.93, 1.53 Hz, 1H)

### EXAMPLE 3

### [2-(2,3-Dichloro-phenyl)-2,4,5,6-tetrahydro-cyclopentapyrazol-3-yl]-pyridin-3-ylmethyl-amine

### EXAMPLE 3A

### 2-(2,3-dichlorophenyl)-2,4,5,6-tetrahydrocyclopenta[c]pyrazol-3-amine

To a suspension of (2,3-dichloro-phenyl)-hydrazine hydrogen chloride (3 g, 10 mmol) in 40 mL ethanol was added 2-oxo-cyclopentanecarbonitrile (purchased from Apollo Scientific) (1 g, 9.2 mmol). The reaction was heated to reflux for 4 hours, cooled and the solvent removed in vacuo. The crude material was taken up in ethyl acetate/water (20/5) and washed with 2M NaOH (1×20 mL) and water (1×20 mL). The combined aqueous layers were extracted with ethyl acetate (2×20 mL) and the combined organics washed with brine (1×40 mL), dried (MgSO₄) and concentrated to give 2.1 g (85%) of a crude solid, which was carried on without further purification. MS (ESI/NH₃) m/z 269 (M+H)⁺;

### EXAMPLE 3B

### N-[2-(2,3-dichlorophenyl)-2,4,5,6-tetrahydrocyclopenta[c]pyrazol-3-yl]nicotinamide

To a solution of Example 3A (0.3 g, 1.19 mmol) and triethylamine (0.2 g, 2 mmol) in 10 mL tetrahydrofuran was added nicotinoyl chloride (0.48 g, 2.68) The reaction was held at room temperature for 18 h, then diluted with ethyl acetate (30 mL). The organics were washed with saturated NaHCO₃ (1×20 mL), water (1×20 mL), brine (1×15 mL), dried (MgSO₄), filtered, and concentrated to give 0.35 g (79%) of a yellow solid. MS (ESI/NH₃) m/z 372 (M+H)⁺; ¹H NMR (5, DMSO-d₆) 2.25-2.4 (m, 2H), 2.65-2.78 (m, 4H), 7.4-7.6 (m, 3H), 7.72-7.8 (dd, 1H), 8.05-8.12 (dd, 1H), 8.7-8.75 (d, 1H), 8.85 (s, 1H), 10.42 (s, 1H).

### EXAMPLE 3C

### [2-(2,3-Dichloro-phenyl)-2,4,5,6-tetrahydro-cyclopentapyrazol-3-yl]-pyridin-3-ylmethyl-amine

The title compound was prepared using the procedure as described in Example 2, except for substituting the product from Example 3B for Example 1C. MS (DCI/NH₃) m/z 359 (M)⁺, 361 (M+2)⁺. ¹H NMR (400 MHz, DMSO-d₆) δ ppm 2.19 (qt, J=7.36 Hz, 2H) 2.38 (t, J=6.75 Hz, 2H) 2.45 (t, J=7.67 Hz, 2H) 4.26 (d, f-6.14 Hz, 2H) 5.85 (t, J=6.14 Hz, 1H) 7.33 (dd, J=7.67, 4.60 Hz, 1H) 7.43 (dd, J=7.98, 1.53 Hz, 1H) 7.48 (t, J=7.98 Hz, 1H) 7.66 (d, J=7.67 Hz, 1H) 7.75 (dd, J=7.67, 1.53 Hz, 1H) 8.43 (dd, J=4.91, 1.53 Hz, 1H) 8.49 (d, J=1.53 Hz, 1H).

### EXAMPLE 4

### [2-(2,3-Dichloro-phenyl-2,4,5,6-tetrahydro-cyclopentapyrazol-3-yl]-(2-methyl-benzyl)-amine

Step A

Example 3A was processed according to the method of Example 1C, except for substituting the product from Example 3A for Example 1B.

Step B

The title compound was prepared using the procedure as described in Example 2, except for substituting the intermediate obtained from Step A for Example 1C. MS (DCI/NH₃) m/z 372 (M)⁺, 374 (M+2)⁺. ¹H NMR (400 MHz, DMSO-d₆) δ ppm 2.20 (qt, J=7.36 Hz, 2H) 2.26 (s, 3H) 2.34 (t, J=7.36, 6.75 Hz, 2H) 2.49 (t, J=7.67, 7.06 Hz, 2H) 4.23 (d, J=5.83 Hz, 2H) 5.69 (t, J=5.83 Hz, 1H) 7.16 (m, 3H) 7.28 (m, 1H) 7.50 (m, 2H) 7.77 (dd, J=7.06, 2.15 Hz, 1H).

### EXAMPLE 5

### [2-(2,3-Dichloro-phenyl)-2,4,5,6-tetrahydro-cyclopentapyrazol-3-yl]-(2-methyl-pyridin-3-ylmethyl)-amine

### EXAMPLE 5A

### N-[2-(2,3-Dichloro-phenyl)-2,4,5,6-tetrahydro-cyclopentapyrazol-3-yl]-2-methyl-nicotinamide

A mixture of Example 3A (268 mg, 1.0 mmol), 2-methyl-nicotinic acid (137 mg, 1.0 mmol), O-(7-azabenzotriazol-1-yl)-N,N,N′,N′-tetramethyluronium hexafluorophosphate (570 mg, 1.5 mmol) and triethylamine (836 μL, 6.0 mmol) in N,N-dimethylformamide (10 mL) was stirred at room temperature overnight. The reaction mixture was poured into water, and extracted with ethyl acetate (2×). The organics were concentrated in vacuo and the resulting residue was chromatographed over silica gel eluting with hexane/ethyl acetate (6:1) then ethyl acetate to give the title compound (195 mg, 50%). MS (DCI/NH₃) m/z 387 (M)⁺, 389 (M+2)⁺.

### EXAMPLE 5B

### [2-(2,3-Dichloro-phenyl)-2,4,5,6-tetrahydro-cyclopentapyrazol-3-yl]-(2-methyl-pyridin-3-ylmethyl)-amine

The title compound was prepared using the procedure as described in Example 2, except for substituting the product from Example 5A for Example 1C. MS (DCI/NH₃) m/z 373 (M)⁺, 375 (M+2)⁺. ¹H NMR (400 MHz, DMSO-d₆) δ ppm 2.16 (qt, J=7.06 Hz, 2H) 2.26 (t, J=7.06, 6.14 Hz, 2H) 2.41 (s, 3H) 2.45 (t, J=7.06 Hz, 2H) 4.21 (d, J=5.83 Hz, 2H) 5.79 (t, J=5.83 Hz, 1H) 7.17 (dd, J=7.67, 4.60 Hz, 1H) 7.47 (s, 1H) 7.48 (dd, J=10.13, 7.98 Hz, 1H) 7.56 (d, J=7.67 Hz, 1H) 7.75 (dd, J=5.83, 3.68 Hz, 1H) 8.29 (dd, J=4.60, 1.23 Hz, 1H).

### EXAMPLE 6

### [2-(2,3-Dichloro-phenyl)-2,6-dihydro-4H-thieno[3,4-c]pyrazol-3-yl]-(2-methyl-pyridin-3-ylmethyl)-amine

### EXAMPLE 6A

### 2-(2,3-Dichloro-phenyl)-2,6-dihydro-4H-thieno[3,4-c]pyrazol-3-ylamine

A mixture of (2,3-dichloro-phenyl)-hydrazine hydrogen chloride (1.0 g, 4.9 mmol) and 4-oxotetrahydrothiophene-3-carbonitrile (purchased from TCI America)(622 mg, 4.9 mmol) in ethanol (30 mL) was refluxed overnight. After removal of the solvent, the resulting residue was basified with Na₂CO₃ (sat.), and extracted with ethyl acetate (2×). The solvent was removed, and the residue was chromatographed using hexane/ethyl acetate (4:1) to give the title compound (1.19 g, 85%). MS (DCI/NH₃) m/z 286 (M)⁺, 288 (M+2)⁺.

### EXAMPLE 6B

### N-[2-(2,3-Dichloro-phenyl)-2,6-dihydro-4H-thieno[3,4-c]pyrazol-3-yl]-2-methyl-nicotinamide

The title compound was prepared using the procedure as described in Example 5A, except for substituting the product from Example 6A for Example 3A. MS (DCI/NH₃) m/z 405 (M)⁺, 407 (M+2)⁺.

### EXAMPLE 6C

### [2-(2,3-Dichloro-phenyl)-2,6-dihydro-4H-thieno[3,4-c]pyrazol-3-yl]-(2-methyl-pyridin-3-ylmethyl)-amine

The title compound was prepared using the procedure as described in Example 2, except for substituting the product from Example 6B for Example 1C. MS (DCI/NH₃) m/z 391 (M)⁺, 393 (M+2)⁺. ¹H NMR (400 MHz, DMSO-d₆) δ ppm 2.41 (s, 3H) 3.54 (s, 2H) 3.76 (s, 2H) 4.25 (d, J=5.83 Hz, 2H) 6.04 (t, J=6.14 Hz, 1H) 7.19 (dd, J=7.36, 4.60 Hz, 1H) 7.51 (d, J=3.99 Hz, 1H) 7.52 (s, 1H) 7.56 (d, J=8.59 Hz, 1H) 7.79 (dd, J=6.44, 2.76 Hz, 1H) 8.30 (dd, J=4.91, 1.23 Hz, 1H).

### EXAMPLE 7

### 5-Benzylamino-1-(2,3-dichloro-phenyl)-1H-pyrazole-4-carbonitrile

### EXAMPLE 7A

### 5-Bromo-1-(2,3-dichloro-phenyl-1H-pyrazole-4-carbonitrile

To the product from Example 1A (4.5 g, 17.8 mmol) in refluxing CHBr₃ (20 mL) was added dropwise t-butylnitrite (6 mL). The mixture was heated at 87° C. for 1 hr, solvent removed in vacuo and the resulting residue was chromatographed on silica gel eluting with CH₂Cl₂ to give the title compound (3.37 g, 60%). MS (DCI/NH₃) m/z 316 (M−1)⁺, 318 (M+1)⁺. ¹H NMR (400 MHz, DMSO-d₆) δ ppm 7.65 (t, J=8.29 Hz, 1H) 7.77 (dd, J=7.98, 1.23 Hz, 1H) 7.98 (dd, J=7.98, 1.23 Hz, 1H) 8.51 (s, 1H).

### EXAMPLE 7B

### 5-Benzylamino-1-(2,3-dichloro-phenyl)-1H-pyrazole-4-carbonitrile

A mixture of the product from Example 7A (50 mg, 0.16 mmol) and benzylamine (500 μL) was heated at 150° C. overnight. The mixture was cooled to room temperature, and purified by preparative high pressure liquid chromatography on a Waters Symmetry C18 column (40 mm×100 mm, 7 μm particle size) using a gradient of 10%:85%: 5% (acetonitrile: H₂O: ammonium acetate) to 95%: 4%: 1% (acetonitrile: H₂O: ammonium acetate) at a flow rate of 40 mL/min. to give the title compound (5.6 mg, 10%). MS (DCI/NH₃) m/z 343 (M)⁺, 345 (M+2)⁺. ¹HNMR (400 MHz, DMSO-d₆) δ ppm 2.54 (s, 3H) 4.52 (d, J=6.75 Hz, 2H) 7.30 (m, 6H) 7.55 (s, 1H) 7.57 (d, J=1.53 Hz, 1H) 7.83 (s, 1H) 7.87 (dd, J=5.52, 3.99 Hz, 1H)

### EXAMPLE 8

### [2-(2,3-Dichloro-phenyl)-2,6-dihydro-4H-thieno[3,4-c]pyrazol-3-yl]-(2-methyl-benzyl)-amine

### EXAMPLE 8A

### N-[2-(2,3-Dichloro-phenyl)-2,6-dihydro-4H-thieno[3,4-c]pyrazol-3-yl]-2-methyl-benzamide

The title compound was prepared using the procedure as described in Example 1C, except for substituting the product from Example 6A for Example 1B. MS (DCI/NH₃) m/z 404 (M)⁺, 406 (M+2)⁺.

### EXAMPLE 8B

### [2-(2,3-Dichloro-phenyl)-2,6-dihydro-4H-thieno[3,4-c]pyrazol-3-yl]-(2-methyl-benzyl)-amine

The title compound was prepared using the procedure as described in Example 2, except for substituting the product from Example 8A for the product from Example 1C. MS (DCI/NH₃) m/z 390 (M)⁺, 392 (M+2)⁺. ¹H NMR (500 MHz, DMSO-d₆) δ ppm 2.23 (s, 3H) 3.53 (s, 2H) 3.75 (s, 2H) 4.22 (d, J=6.10 Hz, 2H) 5.98 (t, J=5.80 Hz, 1H) 7.14 (m, 3H) 7.24 (d, J=6.71 Hz, 1H) 7.50 (s, 1H) 7.52 (s, 1H) 7.79 (t, J=4.88 Hz, 1H)

### EXAMPLE 9

### 1-(2,3-Dichloro-phenyl)-5-[(pyridin-3-ylmethyl)-amino]-1H-pyrazole-4-carbonitrile

The title compound was prepared using the procedure as described in Example 7B, except for substituting C-pyridin-3-yl-methylamine for benzylamine. MS (DCI/NH₃) m/z 344 (M)⁺, 346 (M+2)⁺. ¹H NMR (500 MHz, DMSO-d₆) δ ppm 4.56 (t, J=5.49 Hz, 2H) 7.37 (d, J=4.88 Hz, 1H) 7.39 (d, J=4.58 Hz, 1H) 7.58 (d, J=8.85 Hz, 1H) 7.59 (d, J=3.36 Hz, 1H) 7.69 (dt, J=7.93, 1.83 Hz, 1H) 7.85 (s, 1H) 7.89 (dd, J=7.63, 2.14 Hz, 1H) 8.47 (dd, J=4.58, 1.53 Hz, 1H) 8.52 (d, J=1.83 Hz, 1H)

### EXAMPLE 10

### [2-(2,3-Dichloro-phenyl)-4,5,6,7-tetrahydro-2H-indazol-3-yl]-(2-methyl-pyridin-3-ylmethyl)-amine

### EXAMPLE 10A

### 2-(2,3-Dichloro-phenyl)-4,5,6,7-tetrahadro-2H-indazol-3-ylamine

The title compound was prepared using the procedure as described in Example 6A, except for substituting 2-oxo-cyclohexanecarbonitrile (purchased from Matrix Scientific) for 4-oxotetrahydrothiophene-3-carbonitrile. (DCI/NH₃) m/z 282 (M)⁺, 284 (M+2)⁺.

### EXAMPLE 10B

### N-[2-(2,3-Dichloro-phenyl)-4,5,6,7-tetrahydrho-2H-indazol-3-yl]-2-methyl-nicotinamide

The title compound was prepared using the procedure as described in Example 5A, except for substituting the product from Example 10A for Example 3A. MS (DCI/NH₃) m/z 401 (M)⁺, 403 (M+2)⁺.

### EXAMPLE 10C

### [2-(2,3-Dichloro-phenyl)-4,5,6,7-tetrahydro-2H-indazol-3-yl]-(2-methyl-pyridin-3-ylmethyl-amine

The title compound was prepared using the procedure as described in Example 2, except for substituting the product from Example 10B for Example 1C. MS (DCI/NH₃) m/z 387 (M)⁺, 389 (M+2)⁺. ¹H NMR (400 MHz, DMSO-d₆) δ ppm 1.57 (m, 2H) 1.65 (m, 2H) 2.31 (t, J=6.14 Hz, 2H) 2.32 (s, 3H) 2.45 (t, J=6.14 Hz, 2H) 4.14 (d, J=6.44 Hz, 2H) 5.40 (t, J=6.44 Hz, 1H) 7.14 (dd, J=7.67, 4.91 Hz, 1H) 7.37 (dd, J=7.98, 1.53 Hz, 1H) 7.43 (t, J=7.98 Hz, 1H) 7.51 (d, J=7.36 Hz, 1H) 7.71 (dd, J=7.98, 1.84 Hz, 1H) 8.26 (dd, J=4.60, 1.53 Hz, 1H).

### EXAMPLE 11

### 1-(2,3-Dichloro-phenyl-5-[(2-methyl-pyridin-3-ylmethyl)-amino]-1H-pyrazole-4-carbonitrile

The title compound was prepared using the procedure as described in Example 7B, except for substituting C-(2-methyl-pyridin-3-yl)-methylamine for benzylamine. MS (DCI/NH₃) m/z 358 (M)⁺, 360 (M+2)⁺. ¹H NMR (400 MHz, DMSO-d₆) δ ppm 2.43 (s, 3H) 4.51 (d, J=6.14 Hz, 2H) 7.20 (dd, J=7.67, 4.60 Hz, 1H) 7.33 (t, J=6.14 Hz, 1H) 7.54 (d, J=8.90 Hz, 1H) 7.58 (t, J=7.98 Hz, 1H) 7.64 (dd, J=7.98, 1.53 Hz, 1H) 7.84 (m, 1H) 7.88 (dd, J=7.98, 1.53 Hz, 1H) 8.32 (dd, J=4.60, 1.23 Hz, 1H)

### EXAMPLE 12

### [2-(2,3-Dichloro-phenyl)-2,6-dihydro-4H-thieno[3,4-c]pyrazol-3-yl]-(2-phenoxy-pyridin-3-ylmethyl-amine

### EXAMPLE 12A

### N-[2-(2,3-Dichloro-phenyl)-2,6-dihydro-4H-thieno[3,4-c]pyrazol-3-yl]-2-phenoxy-nicotinamide

2-Phenoxynicotinic acid (Lancaster Synthesis) (512 mg, 2.38 mmol) in CH₂Cl₂ (20 mL) was treated with oxalyl chloride (250 μL). To the mixture was added 2 drops of N,N-dimethylformamide and the reaction mixture was stirred at room temperature for 1 h Solvents were removed in vacuo and the resulting residue was added to a mixture of the product from Example 6A (300 mg, 1.05 mmol), 4-dimethylaminopyridine (50 mg) and triethylamine (2 mL) in tetrahydrofuran (30 mL). The mixture was stirred at room temperature overnight. The solvent was removed and the residue was chromatographed over silica gel eluting with hexane/ethyl acetate (2:1) to give the title compound (220 mg, 43%). MS (DCI/NH₃) m/z 483 (M)⁺, 485 (M+2)⁺.

### EXAMPLE 12B

### [2-(2,3-Dichloro-phenyl)-2,6-dihydro-4H-thieno[3,4-c]pyrazol-3-yl]-(2-phenoxy-pyridin-3-ylmethyl)-amine

The title compound was prepared using the procedure as described in Example 2, except for substituting the product from Example 12A for Example 1C. The crude material was purified by preparative HPLC on a Waters Symmetry C8 column (40 mm×100 mm, 7 μm particle size) using a gradient of 10% to 100% acetonitrile: 0.1% aqueous TFA at a flow rate of 70 mL/min to provide the trifluoroacetic acid salt of the title compound. MS (DCI/NH₃) m/z 469 (M)⁺, 471 (M+2)⁺. ¹H NMR (400 MHz, CD₃OD) δ ppm 3.76 (s, 2H) 3.84 (s, 2H) 4.45 (s, 2H) 7.02 (m, 2H) 7.10 (dd, J=7.36, 5.22 Hz, 1H) 7.19 (t, J=7.36 Hz, 1H) 7.38 (m, 2H) 7.46 (m, 2H) 7.72 (t, J=5.22 Hz, 1H) 7.78 (d, J=7.36 Hz, 1H) 7.97 (d, J=4.91 Hz, 1H)

### EXAMPLE 13

### [2-(2,3-Dichloro-phenyl)-2,4,5,6-tetrahydro-cyclopentapyrazol-3-yl]-(2-phenoxy-pyridin-3-ylmethyl)-amine

### EXAMPLE 13A

### N-[2-(2,3-Dichloro-phenyl)-2,4,5,6-tetrahydro-cyclopentapyrazol-3-yl]-2-phenoxy-nicotinamide

The title compound was prepared using the procedure as described in Example 12A, except for substituting the product from Example 3A for Example 6A. MS (DCI/NH₃) m/z 465 (M)⁺, 467 (M+2)⁺.

### EXAMPLE 13B

### [2-(2,3-Dichloro-phenyl)-2,4,5,6-tetrahydro-cyclopentapyrazol-3-yl]-(2-phenoxy-pyridin-3-ylmethyl)-amine

The title compound was prepared using the procedure as described in Example 2, except for substituting the product from Example 13A for Example 1C. The crude material was purified by preparative HPLC on a Waters Symmetry C8 column (40 mm×100 mm, 7 μm particle size) using a gradient of 10% to 100% acetonitrile: 0.1% aqueous TFA at a flow rate of 70 mL/min to provide the trifluoroacetic acid salt of the title compound. MS (DCI/NH₃) m/z 451 (M)⁺, 453 (M+2)⁺. ¹H NMR (400 MHz, DMSO-d₆) δ ppm 2.20 (qt, J=7.06 Hz, 2H) 2.37 (t, J=7.36, 6.75 Hz, 2H) 2.49 (t, J=7.98 Hz, 2H) 4.34 (brs, 2H) 5.95 (brs, 1H) 7.07 (m, 2H) 7.11 (dd, J=7.36, 4.91 Hz, 1H) 7.20 (tt, J=7.36, 0.92 Hz, 1H) 7.40 (m, 2H) 7.50 (d, J=2.15 Hz, 1H) 7.51 (s, 1H) 7.73 (dd, J=7.36, 1.53 Hz, 1H) 7.78 (dd, J=5.83, 3.68 Hz, 1H) 7.98 (dd, J=4.91, 1.84 Hz, H).

### EXAMPLE 14

### [2-(2,3-Dichloro-phenyl)-2,4,5,6-tetrahydro-cyclopentapyrazol-3-yl]-(2-phenoxy-benzyl) amine

### EXAMPLE 14A

### N-[2-(2,3-Dichloro-phenyl)-2,4,5,6-tetrahydro-cyclopentapyrazol-3-yl]-2-phenoxy-benzamide

The title compound was prepared using the procedure as described in Example 12A, except for substituting the product from Example 3A for Example 6A, and substituting 2-phenoxybenzoic acid (Aldrich) for 2-phenoxynicotinic acid. MS (DCI/NH₃) m/z 464 (M)⁺, 466 (M+2)⁺.

### EXAMPLE 14B

### [2-(2,3-Dichloro-phenyl)-2,4,5,6-tetrahydro-cyclopentapyrazol-3-yl]-(2-phenoxy-benzyl-amine

The title compound was prepared using the procedure as described in Example 2, except for substituting the product from Example 14A for Example 1C. MS (DCI/NH₃) m/z 450 (M)⁺, 452 (M+2)⁺. ¹H NMR (500 MHz, CD₃OD) δ ppm 2.28 (qt, J=7.63, 7.02 Hz, 2H) 2.47 (t, J=7.63, 6.41 Hz, 2H) 2.55 (t, J=7.63, 6.71 Hz, 2H) 4.33 (s, 2H) 6.84 (m, 3H) 7.07 (t, J=7.63 Hz, 1H) 7.10 (t, J=7.32 Hz, 1H) 7.22 (td, J=7.93, 1.53 Hz, 1H) 7.31 (m, 2H) 7.34 (dd, J=7.93, 1.53 Hz, 1H) 7.39 (m, 2H) 7.66 (dd, J=7.93, 1.53 Hz, 1H).

### EXAMPLE 15

### [2-(3-Chloro-phenoxy)-pyridin-3-ylmethyl]-[2-(2,3-dichloro-phenyl)-2,4,5,6-tetrahydro-cyclopentapyrazol-3-yl]-amine

### EXAMPLE 15A

### 2-(3-Chloro-phenoxy)-N-[2-(2,3-dichloro-phenyl)-2,4,5,6-tetrahydro-cyclopentapyrazol-3-yl-]nicotinamide

The title compound was prepared using the procedure as described in Example 12A, except for substituting the product from Example 3A for Example 6A, and substituting 2-(3-chloro-phenoxy)-nicotinic acid (prepared using the procedure as described in Fujiwara, Hidetoshi; Okabayashi, Ichizo; Chem. Pharm. Bull. 1993, 41, 1163) for 2-phenoxynicotinic acid. MS (DCI/NH₃) m/z 500 (M)⁺, 502 (M+2)⁺.

### EXAMPLE 15B

### [2-(3-Chloro-phenoxy)-pyridin-3-ylmethyl]-[2-(2,3-dichloro-phenyl)-2,4,5,6-tetrahydro-cyclopentapyrazol-3-yl]-amine

The title compound was prepared using the procedure as described in Example 2, except for substituting the product from Example 15A for Example 1C. The crude material was purified by preparative HPLC on a Waters Symmetry C8 column (40 mm×100 mm, 7 μm particle size) using a gradient of 10% to 100% acetonitrile: 0.1% aqueous TFA at a flow rate of 70 mL/min to provide the trifluoroacetic acid salt of the title compound. MS (DCI/NH₃) m/z 486 (M)⁺, 488 (M+2)⁺. ¹H NMR (500 MHz, CD₃OD) δ ppm 2.32 (qt, J=7.32 Hz, 2H) 2.51 (m, 2H) 2.59 (t, J=7.32 Hz, 2H) 4.42 (s, 2H) 6.98 (dd, J=8.24, 1.53 Hz, 1H) 7.06 (t, J=1.83 Hz, 1H) 7.12 (dd, J=7.32, 4.88 Hz, 1H) 7.21 (dd, J=7.93, 1.83 Hz, 1H) 7.37 (t, J=8.24 Hz, 1H) 7.43 (m, 2H) 7.69 (dd, J=7.32, 2.44 Hz, 1H) 7.79 (d, J=7.32 Hz, 1H) 7.99 (dd, J=4.88, 1.83 Hz, 1H).

(e) Biological Data

In Vitro Assays

Tissue Culture: Cells of the THP-1 monocytic cell line (American Type Culture Collection, Rockville, Md.) were maintained in the log phase of growth in RPMI medium containing high glucose and 10% fetal calf serum (BRL, Grand Island, N.Y.) according to established procedures (Humphrey and Dubyak, J. Immunol. Vol. 275 pages 26792-26798, 1996). Fresh vials of frozen THP-1 cells were initiated for growth every eight weeks. To differentiate THP-1 cells into a macrophage phenotype, a final concentration of 25 ng/ml of LPS and 10 ng/ml of IFNγ were added to the cells (Humphrey and Dubyak 1996) either for 3 hours for IL-1β release assays or overnight (16 hours) for pore formation studies. 1321N1 cells stably expressing the recombinant human P2X₇ receptor were grown and used according to previously published protocols (Bianchi, et al, Eur. J. Pharmacol. Vol. 376, pages 127-138, 1999; Lynch et al., Mol. Pharmacol. Vol. 56, pages 1171-1181, 1999). For both the pore formation and IL-1β release assays, cell density and viability were routinely assessed prior to each experiment by trypan dye exclusion and cells found to be >90% viable following differentiation.

P2X₇ Mediated Pore Formation. Activation of the P2X₇ receptor induces nonspecific pore formation and eventually cell lysis (Verhoef et al., The Journal of Immunology, Vol. 170, pages 5728-5738, 2003). Accordingly, the inhibitory activity of the antagonists of the present invention can be determined by their capacity to inhibit the agonist-induced pore formation using the fluorescent dye YO-PRO (MW=629) and Fluorometric Imaging Plate Reader (FLIPR, Molecular Devices, Sunnydale, Calif.). Prior to YO-PRO dye addition, the cells are rinsed once in PBS without Mg²⁺ or Ca²⁺ ions, which have been shown to inhibit pore formation (Michel et al., N-S Arch Pharmacol 359:102-109, 1999). The YO-PRO iodide dye (1 mM in DMSO) is diluted to a final concentration of 2 μM in phosphate buffered saline (PBS without Mg²⁺ or Ca²⁺) and then placed on the cells prior to the addition of the agonist BzATP. Since the THP-1 cells are a non-adherent cell line, the cells are washed in PBS and loaded with the dye in a conical tube prior to spinning the cells onto poly-lysine-coated black-walled 96-well plates, which are utilized to reduce light scattering. After the addition of the agonist BzATP (50 μM, the EC₇₀ value for agonist activation), the YO-PRO dye uptake is observed in the FLIPR apparatus equipped with an Argon laser (wavelength=488 nm) and a CCD camera. The intensity of the fluorescence is captured by the CCD camera every 15 seconds for the first 10 minutes of agonist exposure followed by every 20 seconds for an additional 50 minutes with the data being digitally transferred to an interfaced PC. The exposure setting of the camera is 0.25 sec with an f-stop setting of 2. Solutions of antagonist compounds are prepared by serial dilutions of a 10 mM DMSO solution of the antagonist into the buffer solution with the YO-PRO dye. Antagonist compounds are tested for activity over a concentration range from 0.003 to 100 μM. The test compounds are incubated for 10 minutes with the TBP-1 cells at room temperature, after which the cells are stimulated with BzATP and fluorescence measured as described above in the absence of the antagonist. For antagonist activity measurements, the percent maximal intensity is normalized to that induced by 50 μM BzATP and plotted against each concentration of compound to calculate IC₅₀ values and account for plate-to-plate variability.

IL-1β Release: THP-1 cells are plated in 24-well plates at a density of 1×10⁶ cells /well/ml. On the day of the experiment, cells are differentiated with 25 ng/ml LPS and 10 ng/ml final concentration of γIFN for 3 hours at 37° C. Solutions of antagonist compounds are prepared by serial dilutions of a 10 mM DMSO solution of the antagonist into the PBS solution. In the presence of the differentiation media, the cells are incubated with the antagonists of the present invention for 30 minutes at 37° C. followed by a challenge with 1 mM BzATP for an additional 30 minutes at 37° C. Supernatants of the samples are collected after a 5 minute centrifugation in microfuge tubes to pellet the cells and debris and to test for mature IL-1β released into the supernatant using either R & D Systems Human IL-1β ELISA assay or Endogen Human IL-1β ELISA, following the manufacturer's instructions. The maximum IL-1β release at each concentration of test compound is normalized to that induced by BzATP alone to determine the activity of the test compound. Antagonist potency are expressed as the concentration producing a 50% reduction in release of IL-1β or IC₅₀.

The foregoing in vitro assays can be used to demonstrate the activity of compounds of the invention as P2X₇ antagonists.

In Vivo Assays

Determination of Antinociceptive Effect

Animal handling and experimental protocols were approved by the Institutional Animal Care and Use Committee (IACUC) at Abbott Laboratories. For all surgical procedures, animals were maintained under halothane anesthesia (4% to induce, 2% to maintain), and the incision sites were sterilized using a 10% povidone-iodine solution prior to and after surgeries.

CFA model: The capacity of the antagonists to reduce inflammatory hyperalgesia can be evaluated using the complete Freund's adjuvant (CFA) model. In these experiments, animals are subjected to intraplantar injection of CFA 48 hours before administration of the P2X₇ antagonists. Inhibition of thermal hyperalgesia is determined 30 minutes after antagonist administration by observation of paw withdrawal latency and comparison to response of the contralateral paw.

Chunz model: Efficacy in the reduction of neuropathic pain can be evaluated using the L5/L6 spinal nerve tight ligation (Chung) model in rats. In these experiments, spinal nerve ligation is performed 7-14 days prior to assay. Tactile allodynia is induced by application of a von Frey hair 30 minutes after administration of the antagonist. Reduction in tactile allodynia is measured by determination of the paw withdrawal threshold and comparison to the contralateral paw. (Jarvis et al., Proc. Natl. Acad. USA Vol. 99, pages 17179-17184, 2002).

Zymosan Method: Mice are dosed with experimental compounds orally or subcutaneously 30 minutes prior to injection of zymosan. Mice are then injected intraperitonealy with 2 mg/animal of zymosan suspended in saline. Four hours later the animals are euthanized by CO₂ inhalation and the peritoneal cavities lavaged with 2×1.5 mL of ice cold phosphate buffered saline containing 10 units of heparin/ml. For IL-1β determination the samples are spun at 10,000×g in a refrigerated microfuge (4° C.), supernatants removed and frozen until ELISAs (Enzyme Linked Immuno-Assay) are performed. ELISAs are performed according to manufacture's instructions. IL-1β is determined relative to vehicle control (Perretti M. et al., Agents Actions Vol 35(1-2) pages 71-78 (1992); Torok K, et al., Inflamm Res. Vol 44(6) pages 248-252 (1995)).

The foregoing in vivo assays can be used to demonstrate the activity of compounds of the invention to ameliorate chronic inflammatory pain, neuropathic pain and inflammation.

We claim:

1. A compound having formula (I), 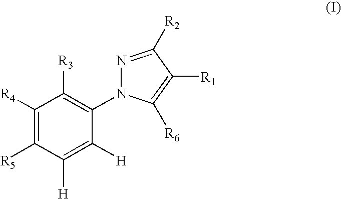
or a pharmaceutically acceptable salt, prodrug, salt of prodrug, or a combination thereof, wherein
R₁ is hydrogen or —CN, and R₂ is hydrogen; or
R₁ and R₂ together with the carbon atoms to which they are attached, form a monocyclic saturated ring consisting of 5, 6 or 7 carbon atoms and one of the carbon atoms of the ring is optionally replaced by a heteroatom selected from the group consisting of S, N, NH, O, SO and SO₂; and said ring is optionally substituted with 1 or 2 substituents selected from the group consisting of alkyl, halogen, haloalkyl, —C(O)alkyl, and —S(O)₂alkyl;
R₃ is halogen, —CN, haloalkyl, alkoxy or haloalkoxy;
R₄ is alkyl, halogen, —CN, haloalkyl, alkoxy or haloalkoxy;
R₅ is hydrogen, alkyl, halogen, —CN, haloalkyl, alkoxy or haloalkoxy;
R₆ is —N(H)—W, or —N(H)—C(Rₓ)(H)—WI; wherein
Rₓ is hydrogen, alkyl or haloalkyl;
W is 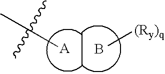
wherein
A is a five or six membered monocyclic ring selected from the group consisting of cycloalkyl and heterocycle and is optionally substituted with 1, 2, or 3 substituents selected from the group consisting of alkyl, halo and haloalkyl;
B is phenyl or monocyclic heteroaryl, optionally substituted with 1, 2 or 3 substituents selected from the group consisting of halo, alkyl, —CN, —OR_{A}, —SR_{A}, —N(R_{A})(R_{B}) and haloalkyl;
q is 0 or 1;
R_{y} is X or -L-X;
W₁ is phenyl or monocyclic heteroaryl, wherein each W₁ is optionally fused with a monocyclic, five or six-membered ring selected from the group consisting of phenyl, heteroaryl, heterocycle, cycloalkyl and cycloalkenyl; wherein each ring as represented by W₁ is independently unsubstituted, substituted with one, two or three R₇, or substituted with zero, one or two R₇ and one substituent selected from the group consisting of X and -L-X;
L at each occurrence is independently O, N(H), N(alkyl), S, S(O), S(O)₂, S(O)₂N(H), SO₂N(alkyl), N(H)S(O)₂, N(alkyl)S(O)₂, CON(H), CON(alkyl), N(H)CO, or N(alkyl)CO);
X, at each occurrence is independently aryl, heteroaryl, cycloalkyl, cycloalkenyl, or heterocycle; each of which is independently unsubstituted or substituted with one, two or three R₇;
R₇ at each occurrence is independently alkyl, alkenyl, CN, NO₂, halo, ═O, —OR_{A}, —SR_{A}, —S(O)R_{A}, —S(O)₂R_{A}, —S(O)₂N(R_{A})(R_{B}), —N(R_{A})(R_{B}), —C(O)R_{A}, —C(O)OR_{A}, —C(O)N(R_{A})(R_{B}), haloalkyl, -alkyl-OR_{A}, -alkyl-SR_{A}, -alkyl-S(O)R_{A}, -alkyl-S(O)₂R_{A}, -alkyl-S(O)₂N(R_{A})(R_{B}), -alkyl-N(R_{A})(R_{B}), -alkyl-C(O)R_{A}, -alkyl-C(O)OR_{A}, or -alkyl-C(O)N(R_{A})(R_{B});
R_{A} at each occurrence is independently hydrogen, alkyl, alkenyl or haloalkyl; and
R_{B} at each occurrence is independently hydrogen, alkyl, or haloalkyl.

2. The compound of claim 1 wherein
R₁ is hydrogen or —CN;
R₂ is hydrogen;
P6 is —N(H)—C(Rₓ)(H)—W₁.

3. The compound of claim 2 wherein
Rₓ is hydrogen;
W₁ is phenyl or monocyclic heteroaryl independently unsubstituted, substituted with one, two or three R₇, or substituted with zero, one or two R₇ and one substituent selected from the group consisting of X and -L-X.

4. The compound of claim 3 wherein
R₁ is hydrogen;
W₁ is substituted phenyl;
R₃ and R₄ are halogen
R₅ is hydrogen and,
R₇ is alkyl.

5. The compound of claim 4 that is
[2-(2,3-Dichloro-phenyl)-2H-pyrazol-3-yl]-(2-methyl-benzyl)-amine

6. The compound of claim 3 wherein
R₁ is —CN;
W₁ is unsubstituted phenyl;
R₃ and R₄ are halogen; and
R₅ is hydrogen.

7. The compound of claim 6 that is
5-Benzylamino-1-(2,3-dichloro-phenyl)-1H-pyrazole-4-carbonitrile.

8. The compound of claim 3 wherein
R₁ is —CN;
R₃ and R₄ are halogen;
R₅ is hydrogen; and
W₁ is unsubstituted monocyclic heteroaryl.

9. The compound of claim 8 that is 1-(2,3-Dichloro-phenyl)-5-[(pyridin-3-ylmethyl)-amino]-1H-pyrazole-4-carbonitrile.

10. The compound of claim 3 wherein
R₁ is —CN;
R₃ and R₄ are halogen;
W₁ is substituted monocyclic heteroaryl; and
R₇ is alkyl.

11. The compound of claim 10 that is
1-(2,3-Dichloro-phenyl)-5-[(2-methyl-pyridin-3-ylmethyl)-amino]-1H-pyrazole-4-carbonitrile

12. The compound of claim 1 wherein
R₁ and R₂ together with the carbon atoms to which they are attached, form a monocyclic saturated ring consisting of 5, 6 or 7 carbon atoms and one of the carbon atoms of the ring is optionally replaced by a heteroatom selected from the group consisting of S, N, NH, O, SO and SO₂; and said ring is optionally substituted with 1 or 2 substituents selected from the group consisting of alkyl, halogen, haloalkyl, —C(O)alkyl, and —S(O)₂alkyl; and
R₆ is —N(H)—C(Rₓ)(H)—W₁.

13. The compound of claim 12, wherein
R₁ and R₂ together with the carbon atoms to which they are attached, form a 5 carbon monocyclic saturated ring;
Rₓ is hydrogen;
R₃ is halogen;
R₄ is halogen; and
W₁ is phenyl or monocyclic heteroaryl.

14. The compound of claim 13, wherein
W₁ is substituted phenyl; and
R₇ is alkyl.

15. The compound of claim 14 that is
[2-(2,3-Dichloro-phenyl)-2,4,5,6-tetrahydro-cyclopentapyrazol-3-yl]-(2-methyl-benzyl)-amine.

16. The compound of claim 13 wherein
W₁ is phenyl substituted with L-X, wherein L is O, and X is aryl.

17. The compound of claim 16 wherein aryl is phenyl.

18. The compound of claim 17 that is
[2-(2,3-Dichloro-phenyl)-2,4,5,6-tetrahydro-cyclopentapyrazol-3-yl]-(2-phenoxy-benzyl)-amine.

19. The compound of claim 13, wherein
W₁ is unsubstituted monocyclic heteroaryl.

20. The compound of claim 10 that is
[2-(2,3-Dichloro-phenyl)-2,4,5,6-tetrahydro-cyclopentapyrazol-3-yl]-pyridin-3-ylmethyl-amine.

21. The compound of claim 13 wherein
W₁ is substituted monocyclic heteroaryl; and
R₇ is alkyl.

22. The compound of claim 21 that is
[2-(2,3-Dichloro-phenyl)-2,4,5,6-tetrahydro-cyclopentapyrazol-3-yl]-(2-methyl-pyridin-3-ylmethyl)-amine.

23. The compound of claim 13 wherein
W₁ is monocyclic heteroaryl substituted with L-X, wherein L is O, and X is aryl.

24. The compound of claim 23 that is selected from the group consisting of
[2-(2,3-Dichloro-phenyl)-2,4,5,6-tetrahydro-cyclopentapyrazol-3-yl]-(2-phenoxy-pyridin-3-ylmethyl)-amine; and
[2-(3-Chloro-phenoxy)-pyridin-3-ylmethyl]-[2-(2,3-dichloro-phenyl)-2,4,5,6-tetrahydro-cyclopentapyrazol-3-yl]-amine.

25. The compound of claim 1 wherein
R₁ and R₂ together with the carbon atoms to which they are attached, form a 5 carbon monocyclic saturated ring and one of the carbon atoms of the ring is replaced by S, SO or SO₂.

26. The compound of claim 25 wherein
R₃ is halogen;
R₄ is halogen;
R₆ is —N(H)—C(Rₓ)(H)—W₁;
Rₓ is hydrogen; and
W₁ is phenyl or monocyclic heteroaryl.

27. The compound of claim 26 wherein W₁ is phenyl substituted with R₇, and R₇ is alkyl.

28. The compound of claim 27, wherein the compound is
[2-(2,3-Dichloro-phenyl)-2,6-dihydro-4H-thieno[3,4-c]pyrazol-3-yl]-(2-methyl-benzyl)-amine.

29. The compound of claim 28 wherein W₁ is monocyclic heteroaryl substituted with R₇, and R₇ is alkyl.

30. The compound of claim 29, wherein the compound is
[2-(2,3-Dichloro-phenyl)-2,6-dihydro-4H-thieno[3,4-c]pyrazol-3-yl]-(2-methyl-pyridin-3-ylmethyl)-amine.

31. The compound of claim 26 wherein
W₁ is monocyclic heteroaryl substituted with L-X, wherein L is O, and X is aryl.

32. The compound of claim 31 that is
[2-(2,3-Dichloro-phenyl)-2,6-dihydro-4H-thieno[3,4-c]pyrazol-3-yl]-(2-phenoxy-pyridin-3-ylmethyl)-amine.

33. The compound of claim 1 wherein
R₁ and R₂ together with the carbon atoms to which they are attached, form a 6 carbon monocyclic saturated ring;
R₃ is halogen;
R₄ is halogen;
R₆ is —N(H)—C(Rₓ)(H)—W₁;
Rₓ is hydrogen; and
W₁ is phenyl or monocyclic heteroaryl.

34. The compound of claim 33, wherein
W₁ is monocyclic heteroaryl substituted with R₇, and R₇ is alkyl.

35. The compound of claim 34 that is
[2-(2,3-Dichloro-phenyl)-4,5,6,7-tetrahydro-2H-indazol-3-yl]-(2-methyl-pyridin-3-ylmethyl)-amine.

36. The compound of claim 1, wherein
R₁ is hydrogen or CN;
R₂ is hydrogen,
R₆ is —N(H)—W, wherein
W is 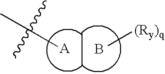
wherein
A is a five or six membered monocyclic ring selected from the group consisting of cycloalkyl and heterocycle and is optionally substituted with 1, 2, or 3 substituents selected from the group consisting of alkyl, halo and haloalkyl; B is phenyl or monocyclic heteroaryl, optionally substituted with 1, 2 or 3 substituents selected from the group consisting of halo, alkyl, —CN, —OR_{A}, —SR_{A}, —N(R_{A})(R_{B}) and haloalkyl; q is 0 or 1; and R_{y} is X or -L-X.

37. The compound of claim 1, wherein R₁ and R₂ together with the carbon atoms to which they are attached, form a monocyclic saturated ring consisting of 5, 6 or 7 carbon atoms and one of the carbon atoms of the ring is optionally replaced by a heteroatom selected from the group consisting of S, N, NH, O, SO and SO₂;
is —N(H)—W, wherein
W is 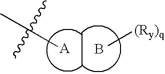
wherein
A is a five or six membered monocyclic ring selected from the group consisting of cycloalkyl and heterocycle and is optionally substituted with 1, 2, or 3 substituents selected from the group consisting of alkyl, halo and haloalkyl; B is phenyl or monocyclic heteroaryl, optionally substituted with 1, 2 or 3 substituents selected from the group consisting of halo, alkyl, —CN, —OR_{A}, —SR_{A}, —N(R_{A})(R_{B}) and haloalkyl; q is 0 or 1; and R_{y} is X or -L-X.

38. A pharmaceutical composition comprising a therapeutically effective amount of a compound of formula (I) as described in claim 1, or a therapeutically acceptable salt, solvate, prodrug, salt of a prodrug, or combination thereof, and a pharmaceutically acceptable carrier.

39. A method of treating or preventing a condition or disorder selected from the group consisting of pain, neuropathic pain, chronic inflammatory pain, inflammation, rheumatoid arthritis, depression and neurodegenerative conditions associated with several progressive CNS disorders, including, but not limited to, Alzheimer's disease, Parkinson's disease, depression, amyotrophic lateral sclerosis, Huntington's disease, dementia with Lewy bodies, multiple sclerosis as well as diminished CNS function resulting from traumatic brain injury, comprising the step of administering a compound of formula (I) as described in claim 1 or a therapeutically acceptable salt, solvate, prodrug, salt of a prodrug, or combination thereof, and a pharmaceutically acceptable carrier.

40. The method of claim 39 wherein the disorder is chronic inflammatory pain or neuropathic pain.

41. The method of claim 39 wherein the disorder is rheumatoid arthritis.

42. The method of claim 39 wherein the disorder is a neurodegenerative condition associated with a CNS disorder including, but not limited to, Alzheimer's disease, Parkinson's disease, depression, amyotrophic lateral sclerosis, Huntington's disease, dementia with Lewy bodies, multiple sclerosis as well as diminished CNS function resulting from traumatic brain injury.

43. The method of claim 39 wherein the disorder is depression.

44. A method for inhibiting P2X₇ activity comprising administering to a patient in need of such treatment a therapeutically effective amount of a compound of formula (I) as described in claim 1, or a therapeutically acceptable salt, solvate, prodrug, salt of a prodrug, or combination thereof.

45. A compound of formula (I) according to claim 1 for use in the manufacture of a medicament for the treatment or prevention of a disease or condition that may be ameliorated by inhibiting P2X₇ receptor activity.
